(19) United States
(12) Patent Application Publication
(10) Pub. No.: US 2014/0093911 A1
(43) Pub. Date: Apr. 3, 2014

(54) METHOD AND APPARATUS FOR IMAGE-BASED PREDICTION AND SORTING OF HIGH-PERFORMING CLONES

(71) Applicants: Tao Sun, Cambridge, MA (US); Joel Voldman, Belmont, MA (US)

(72) Inventors: Tao Sun, Cambridge, MA (US); Joel Voldman, Belmont, MA (US)

(21) Appl. No.: 13/630,399

(22) Filed: Sep. 28, 2012

(51) Int. Cl. C12Q 1/04

(52) U.S. Cl. CPC C12Q 1/04
USPC 435/39; 435/34

(57) ABSTRACT

A method of predictive identification and separation of high-performing cells from a mixed population of cells includes distributing cells belonging to the mixed population to a plurality of open chambers; identifying open chambers containing desired cells and open chambers containing undesired cells; selectively sealing at least one open chamber containing undesired cells; and recovering the desired cells from the open chambers. Cells can be predictively assigned as desired or undesired based on an automated image analysis algorithm.

### CLAIM OF PRIORITY

This application claims priority to provisional U.S. Patent Application No. 61/451,347, filed Sep. 30, 2011, titled “Image-Based Predictive Sorting of High-Performing Clones,” which is incorporated by reference in its entirety.

### TECHNICAL FIELD

This invention relates to a method and apparatus for image-based prediction and sorting of high-performing clones.

### BACKGROUND

Recombinant protein therapeutics are widely used in the treatment of human diseases, ranging from cancer to infertility. Many of these proteins are produced in mammalian cell hosts, which secrete the protein into the culture medium. The traditional procedure to screen and establish a high-producing cell line is time-consuming, involving several rounds of passaging, selection, and evaluation, often for hundreds of different clones. Methods to decrease the time needed for establishing high-producing cell lines would directly reduce pharmaceutical cost.

Two particular issues need to be addressed in selecting a high-performing clone: identification of cells which desirable characteristics for protein production, and separation of those cells from others in a mixed population.

### SUMMARY

A method of predictive identification of high-performing clones relying on image analysis provides benefits in time and expense when developing new cell lines, e.g., cell lines for producing therapeutic biomolecules. Many cells in a mixed population are imaged; the images are analyzed with an algorithm to predict which cells will be high-performing and which will be low performing.

Coupled with this method of predictive identification is a method for separating the high-performing cells from the low-performing cells. Initially the mixed population is distributed in an array of open chambers (e.g., microwells). Imaging and prediction takes place while the cells are in the microwells; and microwells with desired and undesired cells are identified. A polymerizable precursor is deposited over the array, and selective polymerization is carried out to seal the microwells having undesired cells. The undesired cells are thus trapped by the polymer, while the desired cells may be easily recovered by washing. The method allows isolation, imaging, and recovery of single cells in the microwells.

In one aspect, a method of predictive identification of high-performing cells from a mixed population of cells, includes: (a) imaging a cell belonging to the mixed population of cells; (b) determining a value indicative of a phenotypic feature of the cell; (c) repeating step (b) for a plurality of phenotypic features of the cell, thereby providing a phenotypic signature of the cell; (d) predicting a performance metric for the cell based on the phenotypic signature; and (e) repeating steps (a)-(d) for each of a plurality of cells belonging to the mixed population of cells.

Determining a value can include determining a quantitative value. Predicting the performance metric can include using a machine learning algorithm.

The phenotypic features can be selected from the group consisting of:

Cells_Intensity_MinIntensityEdge_aver_Lysosome,

Cells_Intensity_MinIntensity_aver_Lysosome, Cells_Granularity_{—}2_aver_Lysosome,

Cells_Intensity_MaxIntensity_aver_Lysosome,

Cells_Intensity_LowerQuartileIntensity_aver_Lysosome,

Cells_Intensity_MassDisplacement_aver_Nucleus,

Cells_Intensity_MaxIntensity_aver_Nucleus,

Cells_Texture_Entropy_aver_Nucleus_{—}3,

Cells_Texture_DifferenceEntropy_aver_Nucleus_{—}3,

Cells_Texture_SumEntropy_aver_Nucleus_{—}3,

Cells_Granularity_{—}1_aver_Mitochondria,

Cells_RadialDistribution_MeanFrac_aver_Mitochondria_{—}9 of 10,

Cells_RadialDistribution_FracAtD_aver_Mitochondria_{—}6 of 10,

Cells_RadialDistribution_FracAtD_aver_Mitochondria_{—}7 of 10, and

Cells_Intensity_MaxIntensityEdge_aver_Mitochondria.

The method can further include staining the cell with one or more stains selected from the group consisting of a mitochondrial stain, a lysosomal stain, a nuclear stain, a Golgi stain, and an endoplasmic reticulum stain. Each stain can be imaged separately.

The method can further include assigning each cell of the plurality of cells belonging to the mixed population as a desired cell or an undesired cell. The method can further include separating desired cells from undesired cells.

In another aspect, a method of selectively separating cells belonging to a mixed population of cells, includes: (1) distributing cells belonging to the mixed population to a plurality of open chambers; (2) identifying one or more open chambers containing one or more desired cells and one or more open chambers containing one or more undesired cells; (3) selectively sealing the one or more open chambers containing the one or more undesired cells, and leaving open the one or more open chambers containing the one or more desired cells; and (4) recovering the one or more desired cells from the one or more open chambers left open.

The plurality of open chambers can be a plurality of microwells in a microwell array. Selectively sealing can include contacting the microwells with a polymerizable precursor, and selectively polymerizing the precursor at the location of the microwells containing undesired cells. Identifying can include imaging a plurality of cells, assigning each cell of the plurality as a desired cell or an undesired cell, and making a mask corresponding to the locations of the microwells containing desired cells. Recovering the one or more desired cells can include washing the microwell array.

In another aspect, a method of predictive identification and separation of high-performing cells from a mixed population of cells, includes: (1) distributing cells belonging to the mixed population to a plurality of open chambers; (2) identifying one or more open chambers containing one or more desired cells and one or more open chambers containing one or more undesired cells; wherein identifying includes: (a) imaging a cell belonging to the mixed population of cells; (b) determining a value indicative of a phenotypic feature of the cell; (c) repeating step (b) for a plurality of phenotypic features of the cell, thereby providing a phenotypic signature of the cell; (d) predicting a performance metric for the cell based on the phenotypic signature; (e) repeating steps (a)-(d) for each of a plurality of cells belonging to the mixed population of cells; and (f) assigning each cell of the plurality of cells belonging to the mixed population as a desired cell or an undesired cell; (3) selectively sealing the one or more open chambers containing the one or more undesired cells, and leaving open the one or more open chambers containing the one or more desired cells; and (4) recovering the one or more desired cells from the one or more open chambers left open.

Determining a value can include determining a quantitative value. Predicting the performance metric can include using a machine learning algorithm. The phenotypic features can be selected from the group consisting of:

Cells_Intensity_MinIntensityEdge_aver_Lysosome,

Cells_Intensity_MinIntensity_aver_Lysosome, Cells_Granularity_{—}2_aver_Lysosome,

Cells_Intensity_MaxIntensity_aver_Lysosome,

Cells_Intensity_LowerQuartilelntensity_aver_Lysosome,

Cells_Intensity_MassDisplacement_aver_Nucleus,

Cells_Intensity_MaxIntensity_aver_Nucleus,

Cells_Texture_Entropy_aver_Nucleus_{—}3,

Cells_Texture_DifferenceEntropy_aver_Nucleus_{—}3,

Cells_Texture_SumEntropy_aver_Nucleus_{—}3,

Cells_Granularity_{—}1_aver_Mitochondria,

Cells_RadialDistribution_MeanFrac_aver_Mitochondria_{—}9 of 10,

Cells_RadialDistribution_FracAtD_aver_Mitochondria_{—}6 of 10,

Cells_RadialDistribution_FracAtD_aver_Mitochondria_{—}7 of 10, and

Cells_Intensity_MaxIntensityEdge_aver_Mitochondria.

The plurality of open chambers can be a plurality of microwells in a microwell array. Selectively sealing can include contacting the microwells with a polymerizable precursor, and selectively polymerizing the precursor at the location of the microwells containing undesired cells. Identifying can include imaging a plurality of cells, assigning each cell of the plurality as a desired cell or an undesired cell, and making a mask corresponding to the locations of the microwells containing desired cells. Recovering the one or more desired cells can include washing the microwell array.

Other aspects, embodiments, and features will be apparent from the following description, the drawings, and the claims.

### BRIEF DESCRIPTION OF THE DRAWINGS

FIG. 1A schematically depicts a method of developing high performing cell lines, which involves transfection of cells (e.g., mammalian cells), selection of transformants, screening of clones, expansion of clones into larger volumes, and evaluation of clones. This method can require, for example 4-6 months. FIG. 1B schematically depicts a faster method of clone screening, on the order of hours. In FIG. 1B, plated clones are stained and imaged. Based on quantitative image analysis and classification based on staining patterns, cells are classified into according to predicted performance. Clones predicted to be high performing are then sorted and proceed in development.

FIG. 2A shows phase-contrast and fluorescence microscopic images of low-performing (left) and high-performing (right) CHO cell clones. FIG. 2B illustrates image analysis to extract quantitative information from multiple images of cell structures (shown: phase contrast image; mitochondrial fluorescent stain; lysosomal fluorescent stain; and nuclear fluorescent stain). Each cell is assigned values for a variety of image characteristics, including, for example, cell size, shape, texture, granularity, and others.

FIG. 3 is a schematic diagram showing the work flow for applying a support vector machine for classifying low- and high-performing cells.

FIG. 4 is a schematic representation of a method of photoactivated cell sorting.

FIG. 5 is a series of microscopic images of CHO cells from two different clones.

FIG. 6 shows a scatter plot of SVM score vs. a feature value for CHO cells from two different clones.

FIG. 7 is a scatter plot showing SVM scores derived from a set of unknown testing images when each set was recorded from a pure sample of CHO_L or CHO_H.

FIG. 8 shows phase contrast (top) and fluorescence (bottom) images of a mixed sample of CHO_H and CHO_L cells with different stains.

FIG. 9 is scatter plot showing illustrating the successful prediction of CHO_H and CHO_L cells using image based analysis.

FIG. 10 shows scatter plots of SVM scores calculated for cells belonging to each of four different CHO cell clones.

FIG. 11 shows single cell trapping in a microwell array.

FIG. 12 shows single cell isolation in a microwell array by selective sealing of microwells.

FIG. 13 shows images of cells trapped in sealed microwells.

FIG. 14A shows a microwell array where different wells hold different cell types from a mixed population of cells. FIG. 14B shows selective trapping of undesired cells in microwells, where desired cells remain in open wells.

FIGS. 15A-15B shows a microwell array before and after sorting of desired and undesired cells.

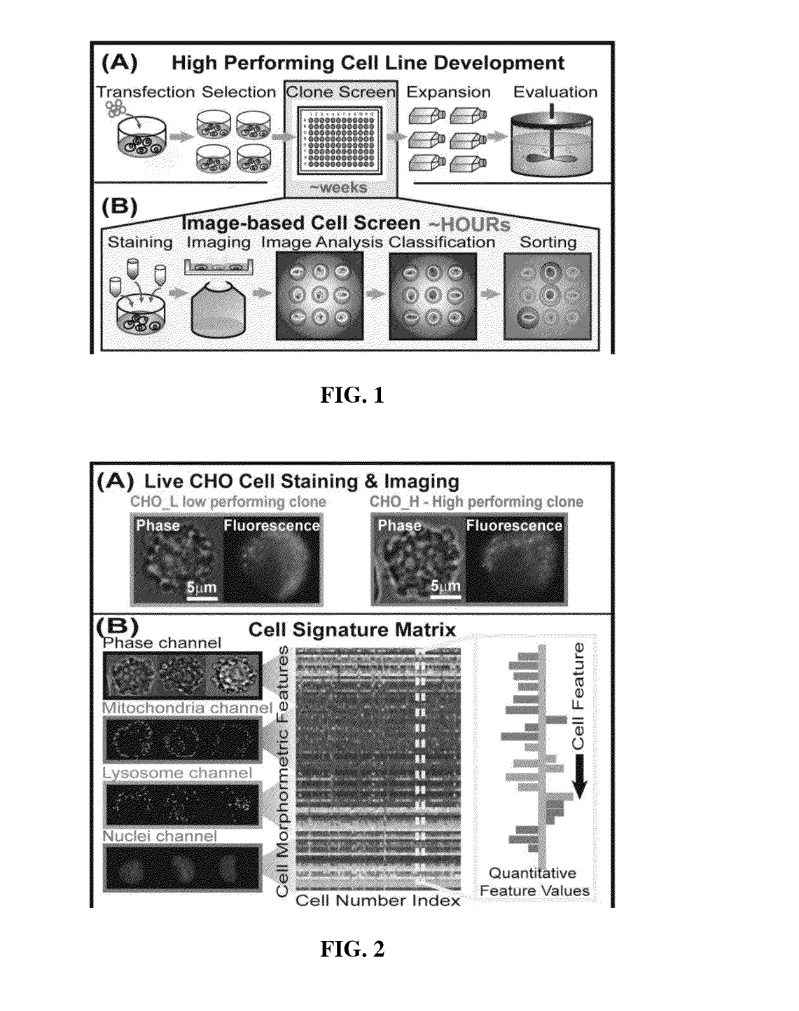

### DETAILED DESCRIPTION

Cell lines are often genetically engineered a desired property to the cells. In one common example, cell lines can be engineered for to produce a desired biomaterial, such a protein, e.g., a therapeutic protein. Development of a new cell line having a desired property can be costly and time-consuming. Initially a population of cells is transformed or transfected with genetic material designed to impart the desired property. That population of cells must then be divided and expanded, to provide a number of clones. The clones must be screened and sorted to isolate those having the desired property.

Among the clones, some will typically display the desired property to varying degrees or with differing qualities. For example, if the desired property is expression of a protein, some clones may produce the protein faster, while others might produce more slowly but for a longer time, providing a greater overall yield. Some clones may produce protein that is misfolded. In some cases, a desired property may be described as the absence of an undesirable property (e.g., a desired property can be the absence of misfolded protein production). Thus, the desired property may not simply be expression of a desired genetic characteristic, but can include more practical concerns as well. Identifying and isolating clones having a more complex set of desired properties, as opposed to a single desired property, can add time, expense, and complexity to the development of a cell line.

The term “desired property” can refer to a single property or a combination of properties. An example given above shows that a desired property may be the combination of rate of protein expression and lifetime.

The term “high-performing clone” can refer to a clone that exhibits a desired property, or combination of desired properties, at a higher level compared to other clones.

The term “low-performing clone” can refer to a clone that exhibits a desired property, or combination of desired properties, at a lower level compared to other clones.

The terms “high-performing” and “low-performing” can be used to describe a binary property (e.g., the presence or absence of a particular feature). The terms “high-performing” and “low-performing” can be used to described a property that exists on a spectrum (e.g., rate of cell division, which can potentially have any value within a range). Where describing a property that exists on a spectrum, the term “high-performing” can refer to a first clone that is high-performing compared to a second, low-performing clone, yet the first clone may be low-performing when compared to a third clone. In some cases, where describing a property that exists on a spectrum, the clones may tend to fall in a bimodal distribution (or other distribution). In that case, all clones tending to fall to one side of the bimodal distribution can be deemed high-performing, and all clones tending to fall to the other side of the bimodal distribution can be deemed low-performing.

A particular clone may be deemed high-performing with respect to one property, but low performing with respect to a different property.

Images of cells convey a wealth of information. Researchers routinely use ubiquitous light microscopes to obtain information about cell structure, shape and motility, cell-cell interactions, and protein expression with spatio-temporal resolution. Because of the wealth of information available, researchers have attempted to use that information to predict cellular properties. Cell fate prediction using image-based phenotyping is applied to a wide range of cell types, including stem cells. See, for example, A. R. Cohen, et al., Nature Methods. 2010, 7, 213; E. M. Chan, et al., Nature Biotechnology. 2009, 27, 1033; and S. Huang, Development. 2009, 136, 3853, each of which is incorporated by reference in its entirety.

The ability to sort cells following such imaging could offer a number of advantages, such as allowing the direct utilization of cells prospectively identified by imaged phenotypes, and allowing investigation into heterogeneity observed via imaging by applying bulk assays to specific cell subpopulations. Unfortunately, facile methods to isolate cells based upon that rich, image-derived information are lacking. FACS offers high throughput, but does not image cells. FACS therefore cannot provide sub-cellular, single-cell, or temporal resolution, and it loses most morphological information because cells are analyzed in suspension. See, e.g., R. G. Ashcroft, P. A. Lopez, J Immunol Methods. 2000, 243, 13, which is incorporated by reference in its entirety. Sorting can be added to microscopy, via live-cell adaptations of laser capture microdis section, laser-based killing of undesired cells, or clone picking following imaging (see, for example, V. Horneffer, et al., Journal of Biomedical Optics. 2007, 12; and M. R. Koller, et al., Cytometry Part A. 2004, 61A, 153, each of which is incorporated by reference in its entirety). However, these platforms have disadvantages such as proprietary culture films, or semi-solid media, and have not been widely adopted.

When developing cell lines, the most visually promising post-transformation clones can be enriched prior to the protracted efforts of dilution cloning. Pooled, barcoded genetic screens, which are now typically limited to phenotypes that can be sorted via fluorescence activated cell sorting (FACS) or that alter proliferation (O. A. Guryanova, et al., Molecular Biology. 2006, 40, 396; and J. Mullenders, et al., Plos One. 2009, 4, each of which is incorporated by reference in its entirety), can be expanded to any phenotypes recognizable through microscopy.

A user-friendly, inexpensive method of sorting cells can utilize photopatternable hydrogels. The method can utilize commercially available reagents and hardware found in most biology labs to create an in-lab photolithography system with rapidly reconfigurable photomasking that enables quick sorting of cells following imaging.

Predictive Identification of High Performing Clones

Images of transfected cells contain phenotypic markers in cellular structures that correlate with various metrics of clone performance. One or more dyes or stains can be used to live-stain a population of transfected cells. The dyes or stains can be chosen to stain different cellular structures (e.g., organelles), such as mitochondria, lysosomes, and nuclei (FIG. 2A).

The stained cells are imaged, e.g., using a wide field fluorescent microscope. The images are quantitatively analyzed to provide a numerical value for a variety of imaged cellular phenotypic features. The number of features can be in the range of, for example, 1 to 1,000, 10 to 500, 20 to 400, or 50 to 250. The cellular phenotypic features measured, can include, for example, cell area, shape, intensity, texture, granularity, and others listed in Table 1.

TABLE 1

| | |
|---|---|
| Cells_AreaShape_Area | Cells_AreaShape_Compactness |
| Cells_AreaShape_Eccentricity | Cells_AreaShape_EulerNumber |
| Cells_AreaShape_Extent | Cells_AreaShape_FormFactor |
| Cells_AreaShape_MajorAxisLength | Cells_AreaShape_MinorAxisLength |
| Cells_AreaShape_Orientation | Cells_AreaShape_Perimeter |
| Cells_AreaShape_Solidity | Cells_Correlation_Correlation_aver_Lysosome_aver_Nucleus |
| Cells_Correlation_Correlation_aver_Mitochondria_aver_Nucleus | Cells_Correlation_Correlation_aver_Mitochondria_aver_Lysosome |
| Cells_Granularity_10_aver_Nucleus | Cells_Granularity_10_aver_Lysosome |
| Cells_Granularity_10_aver_Mitochondria | Cells_Granularity_11_aver_Nucleus |
| Cells_Granularity_11_aver_Lysosome | Cells_Granularity_11_aver_Mitochondria |
| Cells_Granularity_12_aver_Nucleus | Cells_Granularity_12_aver_Lysosome |
| Cells_Granularity_12_aver_Mitochondria | Cells_Granularity_13_aver_Nucleus |
| Cells_Granularity_13_aver_Lysosome | Cells_Granularity_13_aver_Mitochondria |
| Cells_Granularity_14_aver_Nucleus | Cells_Granularity_14_aver_Lysosome |
| Cells_Granularity_14_aver_Mitochondria | Cells_Granularity_15_aver_Nucleus |
| Cells_Granularity_15_aver_Lysosome | Cells_Granularity_15_aver_Mitochondria |
| Cells_Granularity_16_aver_Nucleus | Cells_Granularity_16_aver_Lysosome |
| Cells_Granularity_16_aver_Mitochondria | Cells_Granularity_1_aver_Nucleus |
| Cells_Granularity_1_aver_Lysosome | Cells_Granularity_1_aver_Mitochondria |
| Cells_Granularity_2_aver_Nucleus | Cells_Granularity_2_aver_Lysosome |
| Cells_Granularity_2_aver_Mitochondria | Cells_Granularity_3_aver_Nucleus |
| Cells_Granularity_3_aver_Lysosome | Cells_Granularity_3_aver_Mitochondria |
| Cells_Granularity_4_aver_Nucleus | Cells_Granularity_4_aver_Lysosome |
| Cells_Granularity_4_aver_Mitochondria | Cells_Granularity_5_aver_Nucleus |
| Cells_Granularity_5_aver_Lysosome | Cells_Granularity_5_aver_Mitochondria |
| Cells_Granularity_6_aver_Nucleus | Cells_Granularity_6_aver_Lysosome |
| Cells_Granularity_6_aver_Mitochondria | Cells_Granularity_7_aver_Nucleus |
| Cells_Granularity_7_aver_Lysosome | Cells_Granularity_7_aver_Mitochondria |
| Cells_Granularity_8_aver_Nucleus | Cells_Granularity_8_aver_Lysosome |
| Cells_Granularity_8_aver_Mitochondria | Cells_Granularity_9_aver_Nucleus |
| Cells_Granularity_9_aver_Lysosome | Cells_Granularity_9_aver_Mitochondria |
| Cells_Intensity_IntegratedIntensityEdge_aver_Nucleus | Cells_Intensity_IntegratedIntensityEdge_aver_Lysosome |
| Cells_Intensity_IntegratedIntensityEdge_aver_Mitochondria | Cells_Intensity_IntegratedIntensity_aver_Nucleus |
| Cells_Intensity_IntegratedIntensity_aver_Lysosome | Cells_Intensity_IntegratedIntensity_aver_Mitochondria |
| Cells_Intensity_LowerQuartileIntensity_aver_Nucleus | Cells_Intensity_LowerQuartileIntensity_aver_Lysosome |
| Cells_Intensity_LowerQuartileIntensity_aver_Mitochondria | Cells_Intensity_MassDisplacement_aver_Nucleus |
| Cells_Intensity_MassDisplacement_aver_Lysosome | Cells_Intensity_MassDisplacement_aver_Mitochondria |
| Cells_Intensity_MaxIntensityEdge_aver_Nucleus | Cells_Intensity_MaxIntensityEdge_aver_Lysosome |
| Cells_Intensity_MaxIntensityEdge_aver_Mitochondria | Cells_Intensity_MaxIntensity_aver_Nucleus |
| Cells_Intensity_MaxIntensity_aver_Lysosome | Cells_Intensity_MaxIntensity_aver_Mitochondria |
| Cells_Intensity_MeanIntensityEdge_aver_Nucleus | Cells_Intensity_MeanIntensityEdge_aver_Lysosome |
| Cells_Intensity_MeanIntensityEdge_aver_Mitochondria | Cells_Intensity_MeanIntensity_aver_Nucleus |
| Cells_Intensity_MeanIntensity_aver_Lysosome | Cells_Intensity_MeanIntensity_aver_Mitochondria |
| Cells_Intensity_MedianIntensity_aver_Nucleus | Cells_Intensity_MedianIntensity_aver_Lysosome |
| Cells_Intensity_MedianIntensity_aver_Mitochondria | Cells_Intensity_MinIntensityEdge_aver_Nucleus |
| Cells_Intensity_MinIntensityEdge_aver_Lysosome | Cells_Intensity_MinIntensityEdge_aver_Mitochondria |
| Cells_Intensity_MinIntensity_aver_Nucleus | Cells_Intensity_MinIntensity_aver_Lysosome |
| Cells_Intensity_MinIntensity_aver_Mitochondria | Cells_Intensity_StdIntensityEdge_aver_Nucleus |
| Cells_Intensity_StdIntensityEdge_aver_Lysosome | Cells_Intensity_StdIntensityEdge_aver_Mitochondria |
| Cells_Intensity_StdIntensity_aver_Nucleus | Cells_Intensity_StdIntensity_aver_Lysosome |
| Cells_Intensity_StdIntensity_aver_Mitochondria | Cells_Intensity_UpperQuartileIntensity_aver_Nucleus |
| Cells_Intensity_UpperQuartileIntensity_aver_Lysosome | Cells_Intensity_UpperQuartileIntensity_aver_Mitochondria |
| Cells_RadialDistribution_FracAtD_aver_Nucleus_10of10 | Cells_RadialDistribution_FracAtD_aver_Nucleus_1of10 |
| Cells_RadialDistribution_FracAtD_aver_Nucleus_2of10 | Cells_RadialDistribution_FracAtD_aver_Nucleus_3of10 |
| Cells_RadialDistribution_FracAtD_aver_Nucleus_4of10 | Cells_RadialDistribution_FracAtD_aver_Nucleus_5of10 |
| Cells_RadialDistribution_FracAtD_aver_Nucleus_6of10 | Cells_RadialDistribution_FracAtD_aver_Nucleus_7of10 |
| Cells_RadialDistribution_FracAtD_aver_Nucleus_8of10 | Cells_RadialDistribution_FracAtD_aver_Nucleus_9of10 |
| Cells_RadialDistribution_FracAtD_aver_Lysosome_10of10 | Cells_RadialDistribution_FracAtD_aver_Lysosome_1of10 |
| Cells_RadialDistribution_FracAtD_aver_Lysosome_2of10 | Cells_RadialDistribution_FracAtD_aver_Lysosome_3of10 |
| Cells_RadialDistribution_FracAtD_aver_Lysosome_4of10 | Cells_RadialDistribution_FracAtD_aver_Lysosome_5of10 |
| Cells_RadialDistribution_FracAtD_aver_Lysosome_6of10 | Cells_RadialDistribution_FracAtD_aver_Lysosome_7of10 |
| Cells_RadialDistribution_FracAtD_aver_Lysosome_8of10 | Cells_RadialDistribution_FracAtD_aver_Lysosome_9of10 |
| Cells_RadialDistribution_FracAtD_aver_Mitochondria_10of10 | Cells_RadialDistribution_FracAtD_aver_Mitochondria_1of10 |
| Cells_RadialDistribution_FracAtD_aver_Mitochondria_2of10 | Cells_RadialDistribution_FracAtD_aver_Mitochondria_3of10 |
| Cells_RadialDistribution_FracAtD_aver_Mitochondria_4of10 | Cells_RadialDistribution_FracAtD_aver_Mitochondria_5of10 |
| Cells_RadialDistribution_FracAtD_aver_Mitochondria_6of10 | Cells_RadialDistribution_FracAtD_aver_Mitochondria_7of10 |
| Cells_RadialDistribution_FracAtD_aver_Mitochondria_8of10 | Cells_RadialDistribution_FracAtD_aver_Mitochondria_9of10 |
| Cells_RadialDistribution_MeanFrac_aver_Nucleus_10of10 | Cells_RadialDistribution_MeanFrac_aver_Nucleus_1of10 |
| Cells_RadialDistribution_MeanFrac_aver_Nucleus_2of10 | Cells_RadialDistribution_MeanFrac_aver_Nucleus_3of10 |
| Cells_RadialDistribution_MeanFrac_aver_Nucleus_4of10 | Cells_RadialDistribution_MeanFrac_aver_Nucleus_5of10 |
| Cells_RadialDistribution_MeanFrac_aver_Nucleus_6of10 | Cells_RadialDistribution_MeanFrac_aver_Nucleus_7of10 |
| Cells_RadialDistribution_MeanFrac_aver_Nucleus_8of10 | Cells_RadialDistribution_MeanFrac_aver_Nucleus_9of10 |
| Cells_RadialDistribution_MeanFrac_aver_Lysosome_10of10 | Cells_RadialDistribution_MeanFrac_aver_Lysosome_1of10 |
| Cells_RadialDistribution_MeanFrac_aver_Lysosome_2of10 | Cells_RadialDistribution_MeanFrac_aver_Lysosome_3of10 |
| Cells_RadialDistribution_MeanFrac_aver_Lysosome_4of10 | Cells_RadialDistribution_MeanFrac_aver_Lysosome_5of10 |
| Cells_RadialDistribution_MeanFrac_aver_Lysosome_6of10 | Cells_RadialDistribution_MeanFrac_aver_Lysosome_7of10 |
| Cells_RadialDistribution_MeanFrac_aver_Lysosome_8of10 | Cells_RadialDistribution_MeanFrac_aver_Lysosome_9of10 |
| Cells_RadialDistribution_MeanFrac_aver_Mitochondria_10of10 | Cells_RadialDistribution_MeanFrac_aver_Mitochondria_1of10 |
| Cells_RadialDistribution_MeanFrac_aver_Mitochondria_2of10 | Cells_RadialDistribution_MeanFrac_aver_Mitochondria_3of10 |
| Cells_RadialDistribution_MeanFrac_aver_Mitochondria_4of10 | Cells_RadialDistribution_MeanFrac_aver_Mitochondria_5of10 |
| Cells_RadialDistribution_MeanFrac_aver_Mitochondria_6of10 | Cells_RadialDistribution_MeanFrac_aver_Mitochondria_7of10 |
| Cells_RadialDistribution_MeanFrac_aver_Mitochondria_8of10 | Cells_RadialDistribution_MeanFrac_aver_Mitochondria_9of10 |
| Cells_RadialDistribution_RadialCV_aver_Nucleus_10of10 | Cells_RadialDistribution_RadialCV_aver_Nucleus_1of10 |
| Cells_RadialDistribution_RadialCV_aver_Nucleus_2of10 | Cells_RadialDistribution_RadialCV_aver_Nucleus_3of10 |
| Cells_RadialDistribution_RadialCV_aver_Nucleus_4of10 | Cells_RadialDistribution_RadialCV_aver_Nucleus_5of10 |
| Cells_RadialDistribution_RadialCV_aver_Nucleus_6of10 | Cells_RadialDistribution_RadialCV_aver_Nucleus_7of10 |
| Cells_RadialDistribution_RadialCV_aver_Nucleus_8of10 | Cells_RadialDistribution_RadialCV_aver_Nucleus_9of10 |
| Cells_RadialDistribution_RadialCV_aver_Lysosome_10of10 | Cells_RadialDistribution_RadialCV_aver_Lysosome_1of10 |
| Cells_RadialDistribution_RadialCV_aver_Lysosome_2of10 | Cells_RadialDistribution_RadialCV_aver_Lysosome_3of10 |
| Cells_RadialDistribution_RadialCV_aver_Lysosome_4of10 | Cells_RadialDistribution_RadialCV_aver_Lysosome_5of10 |
| Cells_RadialDistribution_RadialCV_aver_Lysosome_6of10 | Cells_RadialDistribution_RadialCV_aver_Lysosome_7of10 |
| Cells_RadialDistribution_RadialCV_aver_Lysosome_8of10 | Cells_RadialDistribution_RadialCV_aver_Lysosome_9of10 |
| Cells_RadialDistribution_RadialCV_aver_Mitochondria_10of10 | Cells_RadialDistribution_RadialCV_aver_Mitochondria_1of10 |
| Cells_RadialDistribution_RadialCV_aver_Mitochondria_2of10 | Cells_RadialDistribution_RadialCV_aver_Mitochondria_3of10 |
| Cells_RadialDistribution_RadialCV_aver_Mitochondria_4of10 | Cells_RadialDistribution_RadialCV_aver_Mitochondria_5of10 |
| Cells_RadialDistribution_RadialCV_aver_Mitochondria_6of10 | Cells_RadialDistribution_RadialCV_aver_Mitochondria_7of10 |
| Cells_RadialDistribution_RadialCV_aver_Mitochondria_8of10 | Cells_RadialDistribution_RadialCV_aver_Mitochondria_9of10 |
| Cells_Texture_AngularSecondMoment_aver_Nucleus_3 | Cells_Texture_AngularSecondMoment_aver_Lysosome_3 |
| Cells_Texture_AngularSecondMoment_aver_Mitochondria_3 | Cells_Texture_Contrast_aver_Nucleus_3 |
| Cells_Texture_Contrast_aver_Lysosome_3 | Cells_Texture_Contrast_aver_Mitochondria_3 |
| Cells_Texture_Correlation_aver_Nucleus_3 | Cells_Texture_Correlation_aver_Lysosome_3 |
| Cells_Texture_Correlation_aver_Mitochondria_3 | Cells_Texture_DifferenceEntropy_aver_Nucleus_3 |
| Cells_Texture_DifferenceEntropy_aver_Lysosome_3 | Cells_Texture_DifferenceEntropy_aver_Mitochondria_3 |
| Cells_Texture_DifferenceVariance_aver_Nucleus_3 | Cells_Texture_DifferenceVariance_aver_Lysosome_3 |
| Cells_Texture_DifferenceVariance_aver_Mitochondria_3 | Cells_Texture_Entropy_aver_Nucleus_3 |
| Cells_Texture_Entropy_aver_Lysosome_3 | Cells_Texture_Entropy_aver_Mitochondria_3 |
| Cells_Texture_Gabor_aver_Nucleus_3 | Cells_Texture_Gabor_aver_Lysosome_3 |
| Cells_Texture_Gabor_aver_Mitochondria_3 | Cells_Texture_InfoMeas1_aver_Nucleus_3 |
| Cells_Texture_InfoMeas1_aver_Lysosome_3 | Cells_Texture_InfoMeas1_aver_Mitochondria_3 |
| Cells_Texture_InfoMeas2_aver_Nucleus_3 | Cells_Texture_InfoMeas2_aver_Lysosome_3 |
| Cells_Texture_InfoMeas2_aver_Mitochondria_3 | Cells_Texture_InverseDifferenceMoment_aver_Nucleus_3 |
| Cells_Texture_InverseDifferenceMoment_aver_Lysosome_3 | Cells_Texture_InverseDifferenceMoment_aver_Mitochondria_3 |
| Cells_Texture_SumAverage_aver_Nucleus_3 | Cells_Texture_SumAverage_aver_Lysosome_3 |
| Cells_Texture_SumAverage_aver_Mitochondria_3 | Cells_Texture_SumEntropy_aver_Nucleus_3 |
| Cells_Texture_SumEntropy_aver_Lysosome_3 | Cells_Texture_SumEntropy_aver_Mitochondria_3 |
| Cells_Texture_SumVariance_aver_Nucleus_3 | Cells_Texture_SumVariance_aver_Lysosome_3 |
| Cells_Texture_SumVariance_aver_Mitochondria_3 | Cells_Texture_Variance_aver_Nucleus_3 |
| Cells_Texture_Variance_aver_Lysosome_3 | Cells_Texture_Variance_aver_Mitochondria_3 |
| Cytoplasm_AreaShape_Area | Cytoplasm_AreaShape_Compactness |
| Cytoplasm_AreaShape_Eccentricity | Cytoplasm_AreaShape_EulerNumber |
| Cytoplasm_AreaShape_Extent | Cytoplasm_AreaShape_FormFactor |
| Cytoplasm_AreaShape_MajorAxisLength | Cytoplasm_AreaShape_MinorAxisLength |
| Cytoplasm_AreaShape_Orientation | Cytoplasm_AreaShape_Perimeter |
| Cytoplasm_AreaShape_Solidity | Nuclei_AreaShape_Area |
| Nuclei_AreaShape_Compactness | Nuclei_AreaShape_Eccentricity |
| Nuclei_AreaShape_EulerNumber | Nuclei_AreaShape_Extent |
| Nuclei_AreaShape_FormFactor | Nuclei_AreaShape_MajorAxisLength |
| Nuclei_AreaShape_MinorAxisLength | Nuclei_AreaShape_Orientation |
| Nuclei_AreaShape_Perimeter | Nuclei_AreaShape_Solidity |

Quantitative imaging is preferably carried out using an automated image analysis software package, such as, for example, CellProfiler and CellProfiler Analyst (see www.cellprofiler.org; Carpenter A E, et al., (2006) Genome Biology 7:R100.; Kamentsky L, et al. (2011) Bioinformatics 2011; Jones TR, et al., BMC Bioinformatics 2008, 9:482; and Jones T R, et al. (2009) PNAS 106(6):1826-1831; each of which is incorporated by reference in its entirety. CellProfiler and CellProfiler Analyst are built on MatLab (MathWorks). The phenotypic features listed in Table 1 can be measured using the CellProfiler and CellProfiler Analyst software. Other suitable software includes ImageJ (see http://rsbweb.nih.gov/ij/ and the ImageJ user guide at http://rsbweb.nih.gov/ij/docs/guide/index.html, each of which is incorporated by reference in its entirety).

The values measured for the phenotypic features can be combined to provide a phenotypic signature of each individual cell imaged. A multi-dimensional phenotypic signature matrix can be established which contains the signature information across hundreds of cells. One example of such a matrix is illustrated in FIG. 2B.

An initial step in the predictive identification is developing a set of phenotypic features which can discriminate between high- and low-performing clones. This process is illustrated in FIG. 3. To this end, two or more different clones with known performance characteristics are imaged. For example, two clones isolated from the same transfection, where one clone has a high level of expression of a desired protein, and the other clone a low level of expression, can be used. As described above, images of cells can be made, e.g., as phase contrast images, or as fluorescent images of cells dyed with one or more dyes or stains that is specific to a particular cellular structure, such as an organelle. In some cases, the cells are simultaneously stained with more than one organelle-specific stain, and the different stains distinguished by virtue of different fluorescent wavelengths and optical filters. This set of images can constitute a training set of images. Once the images of cells are made the images are analyzed for a variety of phenotypic features and a phenotypic signature developed for the different cells. The phenotypic signatures of high performing cells are compared to the phenotypic signatures of low performing cells.

A number of phenotypic signatures can be developed for a given group of clones, each signature being applicable to a different property. For example, one signature can be developed for expression level of a given protein, another signature can be developed for expression level of a different protein, and another signature can be developed for rate of cell division, and so on.

Depending on the number of phenotypic features that contribute to the phenotypic signatures, and the number of imaged cells, the resulting data set can be a high-dimensional data set. To convert this high-dimensional data set into a simple decision score (i.e., to classify a given cell as high- or low-performing for a given property), a machine-based learning algorithm can be implemented. The machine based learning algorithm can be, for example, a support vector machine which generates a binary classifier based on the phenotypic signatures. Machine learning for scoring image-based phenotypic features is described in, for example, and Jones TR, et al. (2009) PNAS 106(6):1826-1831, which is incorporated by reference in its entirety.

Using the training set of images, each cell is assigned a classification score of ‘−1’ or ‘+1’, respectively (e.g., −1 for cells from the low-performing clone and +1 for cells from the high-performing clone). The SVM algorithm computationally determines an optimal hyper-plane (classifier) in the high-dimensional dataset to best separate the two populations in the training samples.

Then the classifier determined by the SVM is validated. To validate the classifier, a testing set of images is recorded. The testing set is made of images of cells taken from the same clones used in the training set, but are images that have not been analyzed by the SVM. The classifier calculates a score for each individual cell in the testing set. For each individual cell, if its computed score is ≧+1, it is deemed a high-performing cell; whereas if its computed score is ≦−1, it is deemed a low-performing cell. Then the actual categories of each cell are compared to the classifier score. An accurate classifier will correctly deem high-performing cells to be high-performing with a low error rate, and similarly will correctly deem low-performing cells to be low-performing with a low error rate.

Once the classifier has been validated, it can be applied to truly unknown populations of cells, e.g., cells in a mixed population that have not been previously categorized as high- or low-performing.

Image-Based Cell Sorting

Identification of high-performing clones is an important step in development of a cell line, but it is critically related to separating the high-performing clones from low-performing clones with high purity. Where the high-performing clones as identified by prediction based on imaging of a mixed population of cells, as discussed above, the separation by conventional means can be exceedingly difficult.

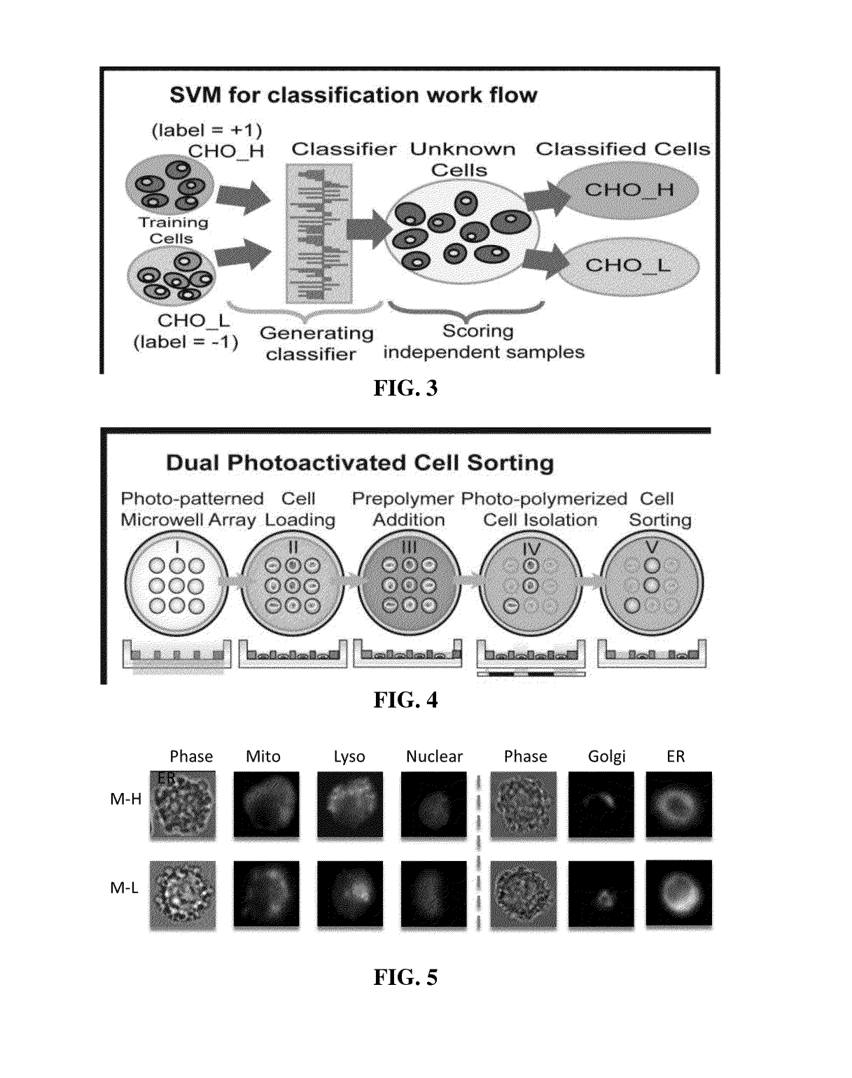

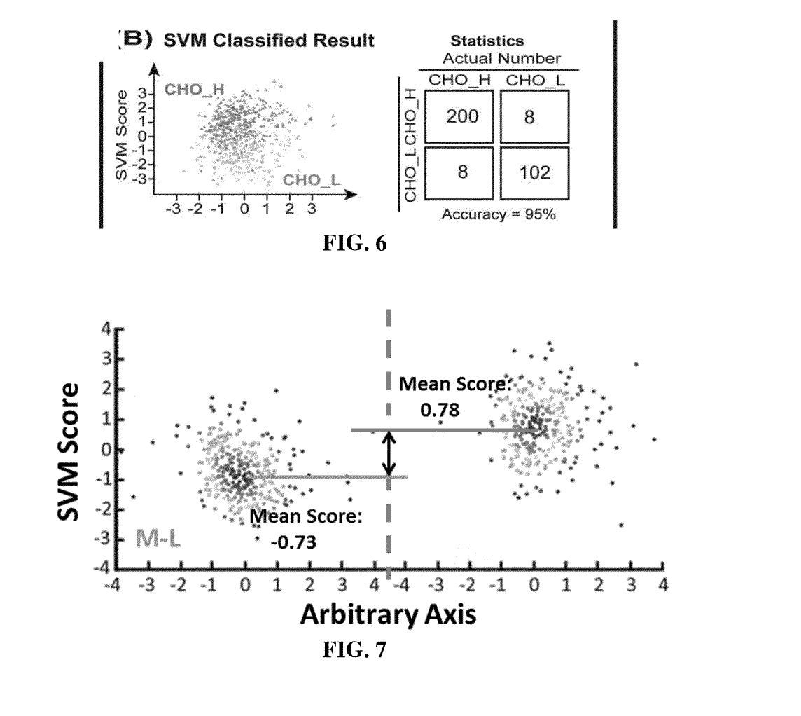

A method of selective image based cell sorting is illustrated in FIG. 5. Briefly, a patterned microwell array is loaded with cells. After imaging and selection of desired cells, a prepolymer is added to the microwell array. A mask is prepared that permits light to expose only those microwells that are to be sealed. After photopolymerization using the mask, the cells in the unsealed microwells can be retrieved.

Instead, separation of clones can be accomplished by first distributing a mixed population of cells in an array of open chambers, such that each open chamber contains a small number of cells. The use of a microwell array is a simple approach for large-scale single-cell trapping. See, for example, J. R. Kovac and J. Voldman, Analytical Chemistry, 2007, 79, 9321-9330; J. C. Love, et al., Nat Biotech, 2006, 24, 703-707; A. Azioune, et al., Lab on a Chip, 2009, 9, 1640-1642; Q. Han, et al., Lab on a Chip, 2010, 10, 1391-1400; and D. K. Wood, et al., Proceedings of the National Academy of Sciences, 2010, 107, 10008-10013. A microwell array can be preferred to random placement of cells on a flat surface because the regular array ensures that cells will be spaced by a known distance (the array spacing), which is can be important for the subsequent sorting step. Additionally, segregating cells in wells should minimize movement of non-adherent cells, which is important for imaging, while also maintaining compatibility with adherent cells.

The number of cells in each open chamber can be controlled to a degree. In general, cells in suspension are deposited over an array of open chambers and allowed to settle randomly in the open chambers. In this method, the number of cells varies from chamber to chamber, and depends on factors such as the density and configuration of open chambers in the array and the concentration of cells in the suspension. If desired, cells can be deposited so that most cells are isolated from all other cells. In this case, some open chambers in the array are likely to have no cells at all, some have one cell exactly, and some, preferably few, of the remaining open chambers have more than one cell. In other cases, cells can be deposited such that most open chambers contain more than one cell. For example, the number of cells in an open chamber can be in the range of 0 to 10, 0 to 5, 0 to 2, or 0 to 1. In some cases, more sophisticated methods of cell manipulation can be used to deposit cells in open chambers. In one example, a single-cell sorting method (e.g., FACS, or micropipette techniques) can be used to deposit cells in open chambers.

The open chambers can be microwells in a microwell array. The microwell array can include polymer walls, positioned on a substrate. In one embodiment, the polymer walls are made by soft imprint lithography (see, e.g., D. Bartolo, Lab on a Chip, 2008, 8, 274-279, which is incorporated by reference in its entirety). In this case, a liquid polymer precursor is deposited on a flat substrate (e.g., of a tissue culture dish). A lithography stamp is contacted to the polymer precursor. Structures on the lithography stamp are recreated in negative when the polymer precursor is polymerized. For example, the stamp can have an array of microposts, which will be rendered as microwells in a polymer slab. The stamp can have any desired microstructure.

Once deposited, the cells in each open chamber are imaged and classified as described above. Based on that classification, a choice is made for each open chamber whether to retain or discard the cell(s) within that open chamber. The open chambers that are to be discarded are then sealed, and the cells in the remaining open chambers (i.e., the desired cells) are retrieved for further development. When desired, the choices in distribution of cells in open chambers and in sealing can allow the retrieval of a single cell from the initial unsorted population of cells. In other cases, a plurality of cells is retrieved, i.e., a population of retrieved cells. The population of retrieved cells is enriched in desired cells compared to the initial unsorted population.

Selected open chambers can be sealed by a selective photopolymerization. A photoactivatable polymer precursor is distributed in the open chambers. The precursor is exposed to light to initiate the photopolymerization; however, those open chambers holding cells to be retrieved are shielded by a mask, such that no photopolymer is formed in those locations. The unpolymerized precursor is removed and the desired cells retrieved. The polymer precursor for sealing can be different than the precursor used in making the polymer walls, and in many cases will be different. The sealing precursor is desirably biocompatible so that the desired cells can be retrieved without harm. The resulting sealing polymer can be a hydrogel. After sealing, the desired cells can be retrieved by washing the open chambers with a suitable culture medium.

### EXAMPLES

### Example 1

### Predictive Identification of High Performing CHO Cell Clones and Image Based Sorting of Those Clones

Two clones of CHO cells that expressed a secreted protein at different levels were used to demonstrate image based predictive sorting. The low-performing clone, CHO_L, expressed the protein at 147 mg/L, whereas the high-performing clone expressed the same protein at 422 mg/L. Different combinations of commercially available dyes to were used to live-stain different cellular organelles of CHO cells, including mitochondria, lysosome and nuclei (FIG. 2A and FIG. 5). CHO_L and CHO_H cells could not be distinguished based on staining patterns by eye alone.

It was found that images of transfected CHO cells contain phenotypic markers in cellular organelles that correlate with various metrics of clone performance. The stained cells were imaged using a wide field fluorescent microscope. An automated image processing and analysis pipeline was developed using the CellProfiler and CellProfiler Analyst software packages to quantitatively extract the phenotypic signature of every individual cell. The signature was composed of the phenotypic features listed in Table 1. A multi-dimensional cell signature matrix was established containing the signature information of over hundreds of cells (FIG. 2B).

Using known datasets of the two cell lines as a training image set, the CHO_L and CHO_H cells were given a classification score of ‘−1’ and ‘+1’, respectively. The SVM algorithm computationally determined an optimal hyper-plane (classifier) in the multi-dimensional data space to best separate the two populations in the training samples. This determined classifier was used to score the testing samples (independent from training samples). For each individual cell, if its computed score is >=+1, we classified it as a high-performing cell, while its computed score is <=−1, we classified it as a low-performing cell. FIG. 6 shows the scatter plot of SVM score vs. a feature value of the pure independent stained CHO_L and CHO_H cells, demonstrating the successful classification of two populations, according to the SVM score distribution. Statistically, the positive prediction rate of each population was 95% accurate. This represents the first demonstration that imaged phenotypes can provide robust information as to clone performance. FIG. 7 shows a scatter plot demonstrating the ability of the classifier to successfully distinguish cells in unknown testing images when each set was recorded from a pure sample of CHO_L or CHO_H.

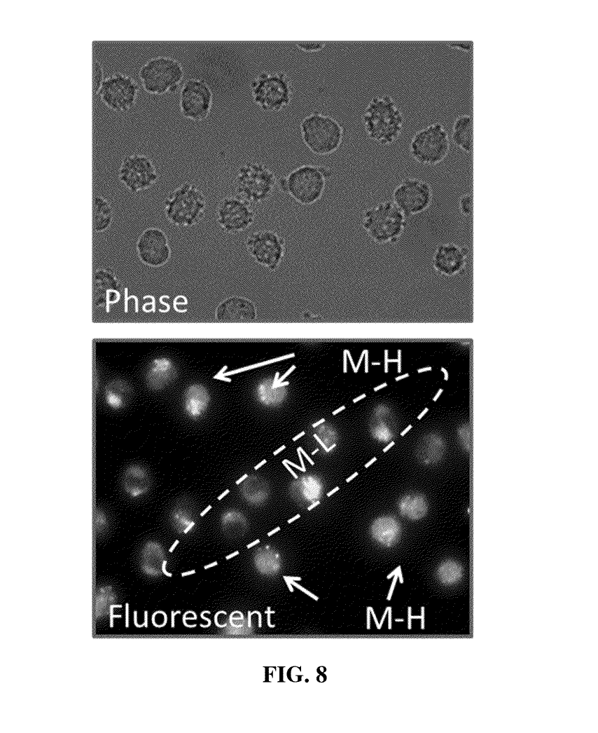

Next the classifier was further validated by using it with a set of images taken from a mixed population of CHO_L and CHO_H cells. The CHO_H cells were stained with a nuclear stain, then after mixing, the mixed sample was stained with a mitochondrial stain and a lyosomal stain, and imaged. FIG. 8 shows phase contrast (top) and fluorescence (bottom) images of the mixed sample. Only the CHO_H cells show nuclear staining (blue). Using only the data from fluorescence of the mitochondrial stain and the lysosomal stain, the classifier was used to predict whether each cell was a CHO_H or a CHO_L cell. Those predictions were then compared to the known identities (determined by the presence or absence of the nuclear stain). FIG. 9 shows a scatter plot showing the intensity of the nuclear stain (distinguishing known CHO_H and known CHO_L cells) plotted against the predicted SVM score. Five regions on the scatter plot are evident: true CHO_H positives at the upper right; true CHO_L positives at the lower left; false CHO_H positives at upper left (i.e., known CHO_L cells incorrectly predicted by the SVM score to be CHO_H cells); and false CHO_L positives at lower right (i.e., known CHO_H cells incorrectly predicted by the SVM score to be CHO_L cells). The fifth region is an undetermined region, including those cells with an SVM score between −1 and +1. The resulting positive prediction rate for CHO_H (i.e., the fraction of cells predicted to be CHO_H that were true positive CHO_H) was 85.1%; for CHO_L, the positive prediction rate was 81.2%.

Certain features were more useful predictors than others. Table 2 shows the phenotypic features of lysosomes, nuclei, and mitochondria that were most predictive.

TABLE 2

| Organelle | Most predictive phenotypic features |
|---|---|
| lysosome | Cells_Intensity_MinIntensityEdge_aver_Lysosome |
| | Cells_Intensity_MinIntensity_aver_Lysosome |
| | Cells_Granularity_2_aver_Lysosome |
| | Cells_Intensity_MaxIntensity_aver_Lysosome |
| | Cells_Intensity_LowerQuartileIntensity_aver_Lysosome |
| nucleus | Cells_Intensity_MassDisplacement_aver_Nucleus |
| | Cells_Intensity_MaxIntensity_aver_Nucleus |
| | Cells_Texture_Entropy_aver_Nucleus_3 |
| | Cells_Texture_DifferenceEntropy_aver_Nucleus_3 |
| | Cells_Texture_SumEntropy_aver_Nucleus_3 |
| mitochondria | Cells_Granularity_1_aver_Mitochondria |
| | Cells_RadialDistribution_MeanFrac_aver_Mitochondria_9of10 |
| | Cells_RadialDistribution_FracAtD_aver_Mitochondria_6of10 |
| | Cells_RadialDistribution_FracAtD_aver_Mitochondria_7of10 |
| | Cells_Intensity_MaxIntensityEdge_aver_Mitochondria |

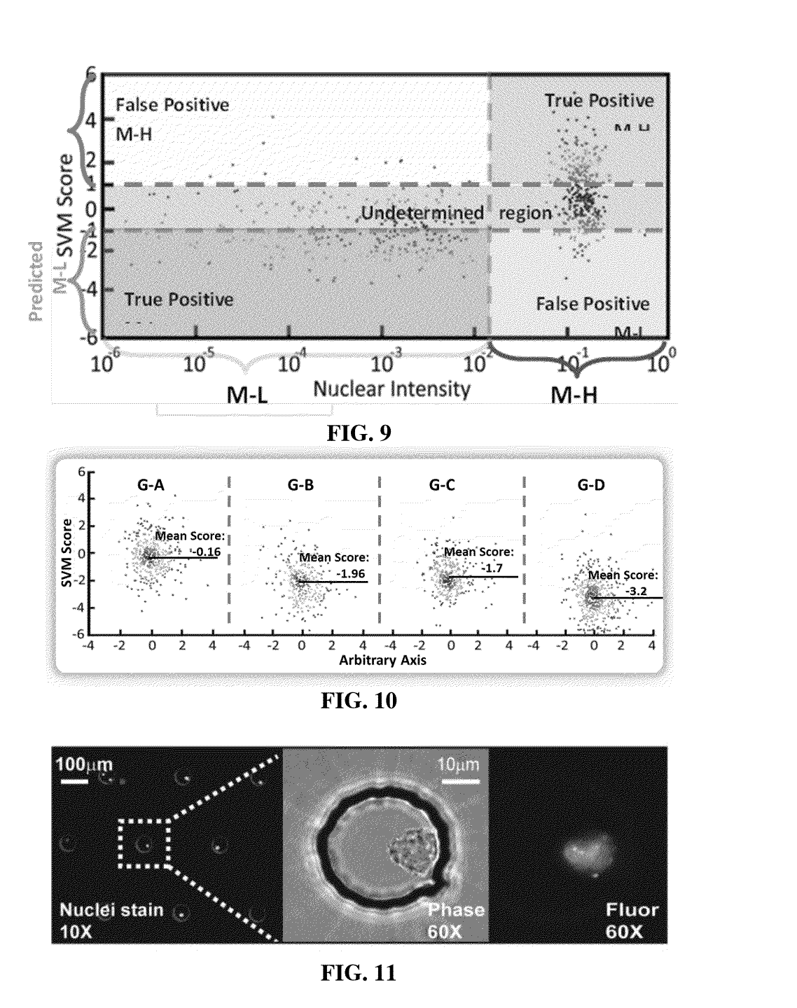

Next, the classifier generated from CHO_H and CHO_L clones was tested for its ability to blindly predict performance of other CHO cell clones. A set of four clones, G-A, G-B, G-C, and G-D, having a range of different performance in secretion of expressed protein were provided by another laboratory. The performance of the different clones was known by the other laboratory but not disclosed until after the image based prediction had been performed. FIG. 10 shows scatter plots of the SVM scores for cells of each of the four clones. The mean SVM scores of the four clones were as follows in Table 3.

TABLE 3

| clone | mean SVM score |
|---|---|
| G-A | −0.16 |
| G-B | −1.96 |
| G-C | −1.7 |
| G-D | −3.2 |

Thus, the predicted relative performance of the clones placed G-A as the highest performing (i.e., greatest SVM score), then G-C, G-B, and G-D as the lowest-performing clone.

When the known rank order of clone performance was unblended, the predicted rank order matched exactly, i.e., G-A>G-C>G-B>G-D. The odds of correctly predicting this result by chance alone was ˜4%.

### Example 2

### Image-Based Sorting of Fluorescent Protein-Expressing Cells

A cell-trapping microwell array was fabricated on the surface of 40-mm-diameter coverslip-bottomed culture dishes (Electron Microscopy Sciences) from the thiolene-based resin NOA 81 (Norland optical adhesive). NOA 81 was chosen instead of polydimethylsiloxane (PDMS) to fabricate the microwell arrays for three main reasons. First, a minimal amount of residual material in the microwell area is desirable to facilitate high resolution microscopic imaging of the cells. The residual layer of NOA 81 during the fabrication can be easily rinsed off using acetone. However, there is no neat way to remove the PDMS residual layer in the microwells. Second, as an adhesive specifically created for optics, exhibit less autofluorescence than PDMS, which is an advantage for imaging. Third, in the second photopolymerization step the photopolymerized sealing polymer can adhere to the NOA 81 polymer better than to PDMS.

To fabricate the microwell array, a droplet (˜25 μl) of photocurable monomer, NOA 81, was deposited on the surface of the glass coverslip. A structured PDMS stamp having microposts (50 μm in diameter and depth, separated by 200 μm) was formed by replica molding from a master, fabricated by lithographically patterning SU-8 photoresist. The PDMS stamp was then gently pressed onto the pre-polymer solution. The diameter and depth of the microposts can be chosen based on the type of cell to be trapped trap specific cell types (see, for example, D. K. Wood, et al., Proceedings of the National Academy of Sciences, 2010, 107, 10008-10013, which is incorporated by reference in its entirety). To cure the pre-polymer (first polymerization), the device was exposed to a collimated 5-cm-diameter ultraviolet (UV) light beam. Briefly, the optical assembly consisted of an X-Cite 120 broadband fluorescence light source (EXFO Inc. USA) and liquid core light guide, an output UG5 filter glass (Thorlabs) to confine the bandpass region of the output light between 240 to 395 nm with an intensity of ˜14.6 mW/cm² at the sample plane as measured by using a UV radiometer (Control Company, USA). An intial photopolymerization was carried out for 7 minutes to partially cure the NOA 81 pre-polymer.

The partially-cured polymer set around the posts and then the PDMS stamp was gently peeled off to form arrayed microwells within the polymer. Acetone (2 ml) was added to the dish for a quick rinse (˜10 seconds) to remove uncured prepolymer. Then 10 ml of deionized (DI) water was added to wash the dish. The cleaned dish was further UV-exposed for 12 minutes to harden the microwell array. After that, the dish was put into an oven (65° C.) for a hard bake overnight; it was then ready to use for single-cell trapping. Typical arrays consisted of 15,000 wells with a well depth of ˜45 μm, as assessed by measuring the height difference between the top and bottom planes of the microwell under the microscopy (Nikon Eclipse TiE automated microscope outfitted with a Ludl BioPrecision 2 motorized stage and Photometrics CoolSnap HQ2 CCD camera). The device allowed the microscopy to be performed using an oil-immersion, high numerical aperture (N.A: 1.4) and short working distance (W.D.: 0.13 mm) objective (Nikon, CFI Plan Apo VC60X) for imaging the cells with high resolution (data not shown). Three global alignment marks were created using a black-color marker pen on the bottom of the dish for subsequently aligning the mask with the dish.

The image-based sorting method was tested using HeLa s3 cells. All HeLa s3 cells were cultured in media containing Dulbecco's Modified Eagle Medium (DMEM) supplemented with 10% v/v fetal bovine serum, 100 U/mL penicillin, 100 μg/mL streptomycin, and 292 μg/mL L-glutamine. One line was generated by infecting HeLa s3 cells with a GFP-CenpA reporter protein, selecting with blasticidin, and dilution-cloned to form a stable cell line. The other cell line was generated by infecting with a mCherry31-NFATc3 construct to form a red fluorescent protein-expressing cell line. Cells were enzymatically dissociated from their culture dishes and loaded into the adhesive-formed microwell array dish at 5×10⁵ cells/ml and allowed to settle under gravity. After the cells settled down (˜5 minutes), untrapped cells not in the microwells were removed by washing the array dish with cell-culture medium.

The next step in the process was to image the cells in the array. The microwell array was scanned under microscopy to image the cell array under phase contrast and fluorescence. Desired cells (here, fluorescent cells) can be determined manually or by automated imaging algorithms. After recording the positions of all the cells of interest, the desired cells must be retrieved while omitting the undesired cells. A parallel, rather than serial, method was desired in order to be able to retrieve many desired cells at once. Since the microwell array was situated in a culture dish, a method that would be compatible with standard pipetting would be advantageous in avoiding enclosed microfluidics. Thus, a method that would allow all desired cells to be retrieved by washing, while encapsulating non-desired cells, was desirable. Photopolymerizable hydrogels were chosen because they are cell-compatible (see, for example, D. Bartolo, et al., Lab on a Chip, 2008, 8, 274-279; A. Revzin, et al., Langmuir, 2003, 19, 9855-9862; and D. R. Albrecht, et al., Lab on a Chip, 2005, 5, 111-118, each of which is incorporated by reference in its entirety), can be configured in real-time simply by changing the polymerization mask, and were found to adhere well to the underlying NOA microwell array.

(Poly) ethylene glycol diacrylate (PEGDA) was used for the second photopolymerization. First, 1 ml of pre-polymer solution was added into the array dish, covering the entire microwell array. The pre-polymer solution consisted of cell culture medium, 20% w/v PEGDA solution (MW 1000, Laysan Bio), 0.2% w/v photo-initiator (Irgacure 2959), 1.6% v/v methanol, and 11,000 U/mL catalase (Sigma). The catalase was used to improve the cell viability. Based on the recorded positions of desired cells, a photo-mask image file was generated consisting of black dots (180 μm in diameter) at desired locations via a Matlab script (Mathworks, USA). Each black dot corresponded to the position of a desired cell. Three alignment circles corresponded to the positions of three global alignment marks on the bottom of the dish. The mask image was printed on a standard inkjet transparency film (Office Depot®, USA) using an inkjet printer (HP deskjet 6540). The mask was then aligned under the array dish by hand under a microscope (Stemi 2000-C, Zeiss). Once the centers of three global alignment marks on the bottom of the dish were simultaneously matched with the centers of three alignment circles on the mask, each black dot was positioned to its corresponding desired cell. Masks were aligned within 1 minute with typical alignment accuracies of 10 μm. After the alignment, the device was exposed to the UV light beam for the second photopolymerization for 10 minutes. After exposure, the PEGDA pre-polymer crosslinked into a hydrogel in all unmasked regions, encapsulating the undesired cells. The thickness of the photoploymerized PEGDA gel was ˜500 μm, estimated by the ratio between the volume of polymerized PEGDA prepolymer and the surface area of the dish. The black dots on the mask shielded the desired cells from the UV exposure, leaving unpolymerized pre-polymer around those cells and thus making them available for retrieval by washing. Finally, we rinsed the dish with cell culture medium and retrieved the desired cells for downstream analysis.

FIG. 11 shows that a microwell array can be used to trap single cells, i.e., that one cell can be confined in a microwell separate from any other cells. The left panel shows several microwells imaged by fluorescence, where the trapped cells have been stained with a nuclear stain. The middle panel shows an enlarged phase contrast image of a trapped cell, and the right panel shows the same single cell under fluorescence.

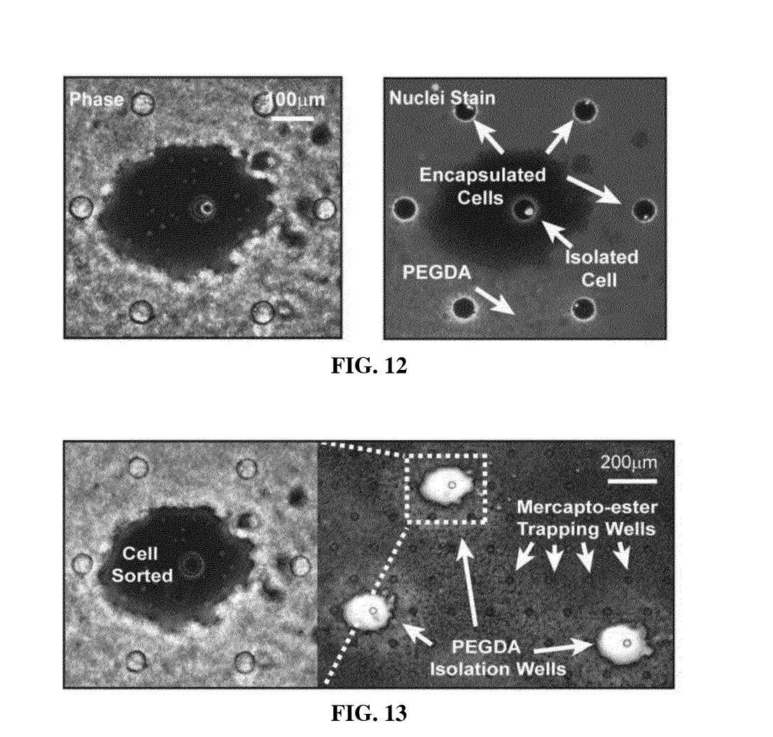

FIG. 12 illustrates isolation of single cells by selective sealing microwells. The left panel shows a phase contrast image of several microwells. The microwell at the center of the image includes a single cell. The other microwells have been sealed by photopolymerization of PEGDA, while the microwell at center was selectively masked from photopolymerization. The right panel, taken in fluorescence, shows that while some cells are encapsulated under PEGDA, the cell in the microwell at center is isolated in an unsealed well.

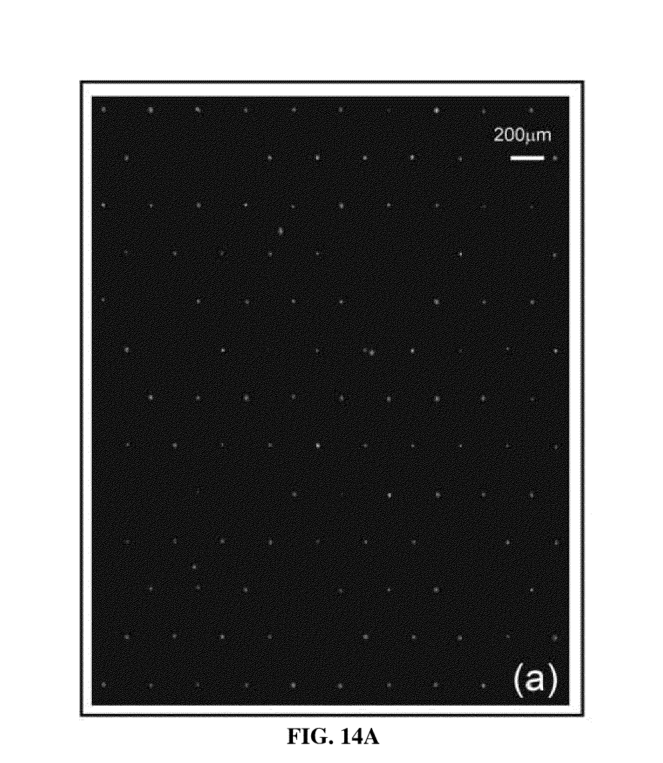

FIG. 13 shows close-up (left) and zoomed out images of PEGDA isolation wells after cell sorting showing that the cells in the unsealed isolation wells have been removed by washing; cells in the sealed trapping wells remain trapped.

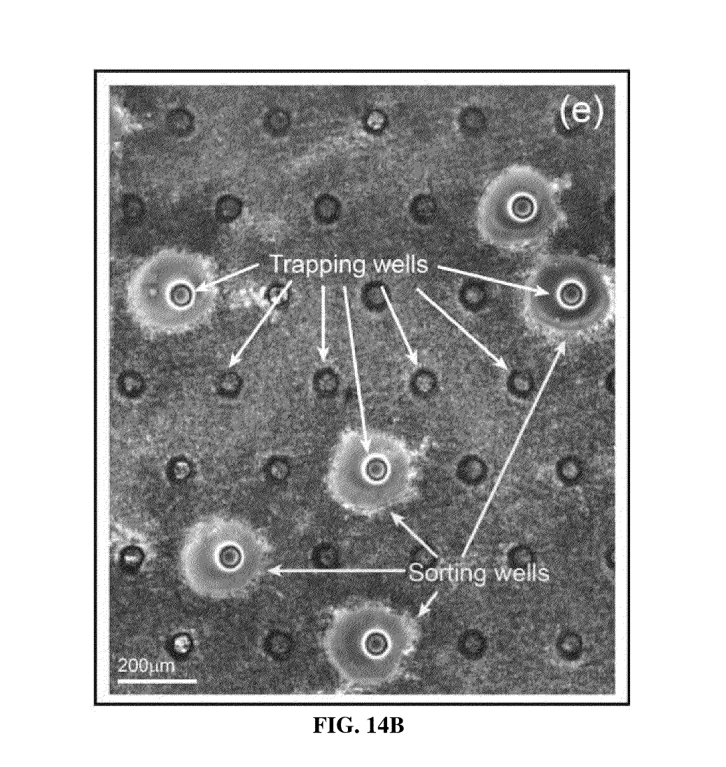

To illustrate and quantitatively characterize the sorting method, whole-cell fluorescence-based sorting was demonstrated. FIG. 14A shows a typical cell trapping result where we loaded a 1:1 ratio of GFP:mCherry-expressing cells to achieve approximately 90% well-filling efficiency. After washing, very few (<1%) cells remained outside the microwells. These results were consistent with prior work on loading microwell arrays (see D. K. Wood, et al., Proceedings of the National Academy of Sciences, 2010, 107, 10008-10013, which is incorporated by reference in its entirety).

To demonstrate the sorting of minority populations (e.g. rare cell isolation), GFP- and mCherry-expressing cells were mixed at a ratio of 1:100 and targeted to sort the rare GFP-expres sing cells, while treating RFP-expres sing cells as undesired cells. First, the desired GFP-expressing cells were targeted via microscopy. The desired GFP-expressing cells were isolated from surrounding mCherry-expressing cells by the photopolymerized PEGDA, leaving the undesired mCherry-expressing cells encapsulated in the cross-linked PEGDA gel. Finally, the desired GFP-expres sing cells were retrieved by simply washing the array, leaving behind the undesired mCherry-expressing cells remaining. The supernatant was collected to retrieve the desired GFP-expressing cells. In the supernatant some undesired mCherry-expressing cells encapsulated in gel were observed, which were likely due to broken fragments of the gel during the washing. Although the gel firmly attached to the surface of the NOA microwell array, it was not as strongly attached to the surface of glass coverslip substrate, resulting in some broken fragments. However, since the majority of fragments were larger than 20 μm whereas the cell size was less than 20 μm, filtering the supernatant through a 20 μm filter removed gel-encapsulated undesired cells and gel debris (>20 μm). Further removal of gel fragments occurred when refreshing the culture medium while feeding the subsequently cultured cells.

FIG. 14B shows a view of 1 mm×1.4 mm region of the array after the desired cells were sorted. The two layers of microwells were evident: the trapping wells made from the photopolymerized optical adhesive, and the sorting wells made from spaces in the photopolymerized PEGDA hydrogel (i.e., where the mask had prevented photopolymerization). The size of the sorting wells was dictated by the size of the trapping wells and the spacing between the two adjacent trapping wells. To assess the recovery and purity of sorted cells, 41 microwells that contained desired GFP-expressing cells were selected, and those cells selectively retrieved as described. After sorting, the retrieved cells were plated into one well of a 96-well plate and monitored their growth in culture. After sorting, 3 colonies of mCherry-expressing cells and 32 colonies of GFP-expressing cells were observed. This result corresponds to a recovery of >75% and a purity of >90%. The undesired mCherry-expressing cells possibly came from targeted microwells containing both a GFP-expressing cell and a mCherry-expressing cell. Cell loss (˜25%) happened during the dish washing, supernatant filtering, cell centrifuging and re-plating steps. In all, the concentration of desired cells in the population was enhanced by ˜3 orders of magnitude.

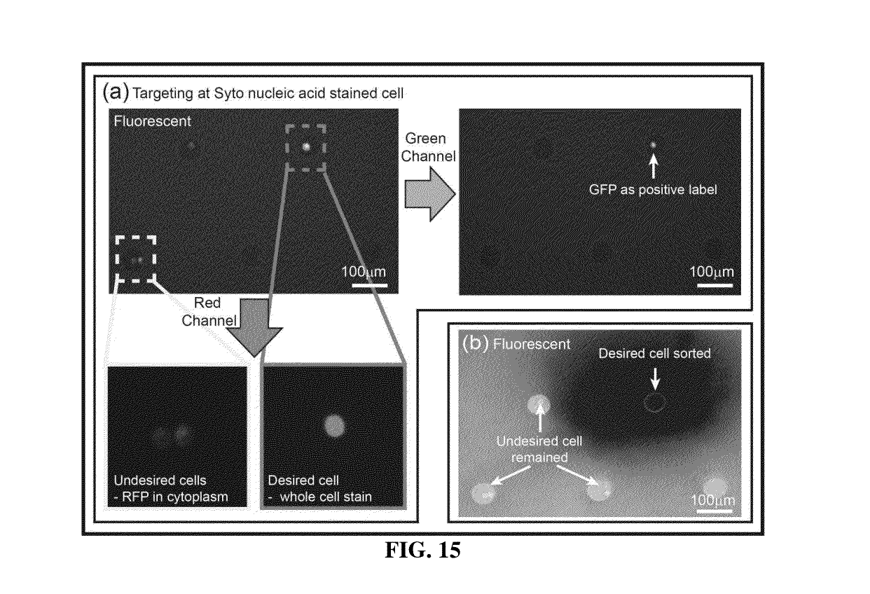

To demonstrate sorting of a phenotype that cannot be achieved by FACS, cells were sorted based on localization of fluorescence, rather than the presence or absence of a particular fluorescence wavelength. GFP-expressing cells were stained with SYTO 82 Orange nucleic acid dye (0.5 μM concentration, 5 minutes, Invitrogen). The red-dye stained GFP-expressing cells were then mixed with the mCherry-expressing cells at a ratio of 1:100. The SYTO-stained GFP-expressing cells (desired) showed whole-cell red fluorescence because the SYTO dye stains both DNA and (cytoplasmic) RNA, while the mCherry-expressing cells (undesired) showed red fluorescence only within the cytoplasm because the mCherry was fused to NFATc3, which is normally resident in the cytoplasm. Microscopic imaging was used to distinguish the cells that had red fluorescence throughout the cell (desired) versus those where the red fluorescence was only in the cytoplasm (undesired), and used green fluorescent as a “ground truth” to verify the assessment, as shown in FIG. 15A. FIG. 15B shows a closeup of the array demonstrating that the desired SYTO-stained GFP-expressing cell was removed, while the mCherry-expressing cells remained.

Other embodiments are within the scope of the following claims.

1. A method of predictive identification of high-performing cells from a mixed population of cells, comprising:
(a) generating a cellular image of a cell belonging to the mixed population of cells;
(b) determining a value indicative of a phenotypic feature of the cell based on the cellular image of the cell;
(c) repeating step (b) for a plurality of phenotypic features of the cell, thereby providing a phenotypic signature of the cell;
(d) predicting a performance metric for the cell based on the phenotypic signature; and
(e) repeating steps (a)-(d) for each of a plurality of cells belonging to the mixed population of cells.

2. The method of claim 1, wherein determining a value includes determining a quantitative value.

3. The method of claim 1, wherein predicting the performance metric includes using a machine learning algorithm.

4. The method of claim 1, wherein the phenotypic features are determined from area, shape, intensity, texture, or granularity of a cell, lysosome, nucleus, or mitochondrion.

5. The method of claim 1, further comprising staining the cell with one or more stains selected from the group consisting of a mitochondrial stain, a lysosomal stain, a nuclear stain, a Golgi stain, and an endoplasmic reticulum stain.

6. The method of claim 5, wherein each stain is imaged separately.

7. The method of claim 1, further comprising assigning each cell of the plurality of cells belonging to the mixed population as a desired cell or an undesired cell.

8. The method of claim 7, further comprising separating desired cells from undesired cells.

9. A method of selective cellular imaging based sorting of desired cells belonging to a mixed population of cells, wherein the desired cells have been predictively identified based on cellular imaging, the method comprising:
(1) distributing cells belonging to the mixed population to a plurality of open chambers;
(2) identifying one or more open chambers containing one or more desired cells and one or more open chambers containing one or more undesired cells;
(3) selectively sealing the one or more open chambers containing the one or more undesired cells, and leaving open the one or more open chambers containing the one or more desired cells; and
(4) recovering the one or more desired cells from the one or more open chambers left open.

10. The method of claim 9, wherein the plurality of open chambers is a plurality of microwells in a microwell array.

11. The method of claim 10, wherein selectively sealing includes contacting the microwells with a polymerizable precursor, and selectively polymerizing the precursor at the location of the microwells containing undesired cells.

12. The method of claim 9, wherein identifying includes cellular imaging a plurality of cells, assigning each cell of the plurality as a desired cell or an undesired cell, and making a mask corresponding to the locations of the microwells containing desired cells.

13. The method of claim 9, wherein recovering the one or more desired cells includes washing the microwell array.

14. A method of predictive identification and separation of high-performing cells from a mixed population of cells, comprising:
(1) distributing cells belonging to the mixed population to a plurality of open chambers;
(2) identifying one or more open chambers containing one or more desired cells and one or more open chambers containing one or more undesired cells; wherein identifying includes:
(a) generating a cellular image of a cell belonging to the mixed population of cells;
(b) determining a value indicative of a phenotypic feature of the cell based on the cell's cellular image;
(c) repeating step (b) for a plurality of phenotypic features of the cell, thereby providing a phenotypic signature of the cell;
(d) predicting a performance metric for the cell based on the phenotypic signature;
(e) repeating steps (a)-(d) for each of a plurality of cells belonging to the mixed population of cells; and
(f) assigning each cell of the plurality of cells belonging to the mixed population as a desired cell or an undesired cell, based on the predictive performance metric for the cell;
(3) selectively sealing the one or more open chambers containing the one or more undesired cells, and leaving open the one or more open chambers containing the one or more desired cells; and
(4) recovering the one or more desired cells from the one or more open chambers left open.

15. The method of claim 14, wherein determining a value includes determining a quantitative value.

16. The method of claim 14, wherein predicting the performance metric includes using a machine learning algorithm.

17. The method of claim 14, wherein the phenotypic features are determined from area, shape, intensity, texture, or granularity of a cell, lysosome, nucleus, or mitochondrion.

18. The method of claim 14, wherein the plurality of open chambers is a plurality of microwells in a microwell array.

19. The method of claim 14, wherein selectively sealing includes contacting the microwells with a polymerizable precursor, and selectively polymerizing the precursor at the location of the microwells containing undesired cells.

20. The method of claim 14, wherein identifying includes making a mask corresponding to the locations of the microwells containing desired cells.

21. The method of claim 20, wherein recovering the one or more desired cells includes washing the microwell array.
